(19) United States
(12) Patent Application Publication
(10) Pub. No.: US 2017/0055814 A1
(43) Pub. Date: Mar. 2, 2017

(54) Reconstruction of Images from an in Vivo Multi-Camera Capsule with Confidence Matching

(71) Applicant: CAPSOVISION,INC., Saratoga, CA (US)

(72) Inventors: Kang-Huai WANG, Saratoga, CA (US); Chenyu WU, Sunnyvale, CA (US)

(73) Assignee: CAPSOVISION ,INC., Saratoga, CA (US)

(21) Appl. No.: 15/306,475

(22) PCT Filed: Apr. 27, 2015
(86) PCT No.: PCT/US2015/027813

§ 371 Date: Oct. 24, 2016

Related U.S. Application Data
(60) Provisional application No. 62/006,257, filed on Jun. 1, 2014.

(51) Int. Cl. A61B 1/04; G06K 9/52; G06K 9/62; G06T 11/60; A61B 1/00; G06T 7/00

(52) U.S. Cl. CPC A61B 1/041; A61B 1/0005; A61B 1/00009; G06T 7/0012; G06K 9/6201; G06T 11/60; G06K 9/52; G06T 2207/30092; G06T 2207/20076; G06T 2207/10068

(57) ABSTRACT

A method of adaptively displaying images captured using a camera according to quality of image matching. The system utilizes the quality of image matching to guide whether to stitch underlying images or not. The quality of image matching is measured between two images to be matched. The image is declared as a high-confidence image or a low-confidence image according to the quality of image matching. The high-confidence images are stitched into one or more larger composite pictures and displayed on a display device. On the other hand, the low-confidence images are displayed as individual images without stitching.

### CROSS REFERENCE TO RELATED APPLICATIONS

The present invention is related to U.S. Provisional Patent Application Ser. No. 61/828,653, entitled “Reconstruction of Images from an in vivo Multi-Cameras Capsule”, filed on May 29, 2013. The U.S. Provisional Patent Application is hereby incorporated by reference in its entirety.

### FIELD OF THE INVENTION

The present invention relates to image stitching from images captured using in vivo capsule camera and their display thereof.

### BACKGROUND AND RELATED ART

Capsule endoscope is an in vivo imaging device which addresses many of problems of traditional endoscopes. A camera is housed in a swallowable capsule along with a radio transmitter for transmitting data to a base-station receiver or transceiver. A data recorder outside the body may also be used to receive and record the transmitted data. The data primarily comprises images recorded by the digital camera. The capsule may also include a radio receiver for receiving instructions or other data from a base-station transmitter. Instead of using radio-frequency transmission, lower-frequency electromagnetic signals may be used. Power may be supplied inductively from an external inductor to an internal inductor within the capsule or from a battery within the capsule. In another type of capsule camera with on-board storage, the captured images are stored on-board instead of transmitted to an external device. The capsule with on-board storage is retrieved after the excretion of the capsule. The capsule with on-board storage provides the patient the comfort and freedom without wearing the data recorder or being restricted to proximity of a wireless data receiver.

While forward-looking capsule cameras include one camera, there are other types of capsule cameras that use multiple cameras to provide side view or panoramic view. A side or reverse angle is required in order to view the tissue surface properly. It is important for a physician or diagnostician to see all areas of these organs, as polyps or other irregularities need to be thoroughly observed for an accurate diagnosis. A camera configured to capture a panoramic image of an environment surrounding the camera is disclosed in U.S. patent application Ser. No. 11/642,275, entitled “In vivo sensor with panoramic camera” and filed on Dec. 19, 2006.

In an autonomous capsule system, multiple images along with other data are collected during the course when the capsule camera travels through the gastrointestinal (GI) tract. The images and data after being acquired and processed are usually displayed on a display device for a diagnostician or medical professional to examine. However, each image only provides a limited view of a small section of the GI tract. It is desirable to form a large picture from multiple capsule images representing a single composite view. For example, multiple capsule images may be used to form a cut-open view of the inner GI tract surface. The large picture can take advantage of the high-resolution large-screen display device to allow a user to visualize more information at the same time. The image stitching process may involve removing the redundant overlapped areas between images so that a larger area of the inner GI tract surface can be viewed at the same time as a single composite picture. In addition, the large picture can provide a complete view or a significant portion of the inner GI tract surface. It should be easier and faster for a diagnostician or a medical professional to quickly spot an area of interest, such as a polyp.

In the field of computational photography, image mosaicing techniques have been developed to stitch smaller images into a large picture. A review of general technical approaches to image alignment and stitching can be found in “Image Alignment and Stitching: A Tutorial”, by Szeliski, Microsoft Research Technical Report MSR-TR-2004-92, Dec. 10, 2006.

For image mosaicing, corresponding parts, objects or areas among images are identified first. After corresponding parts, objects or areas are identified, in other words, after two images are registered, they can be stitched by aligning the corresponding parts, objects or areas. Two images can be registered directly in the pixel domain or matched based on features extracted from images. The pixel-based image matching is also called direct match. There are several similarity measurements that can be used for evaluating the quality of image matching, such as sum of squared distance (SSD), normalized cross correlation (NCC), mutual information (MI) etc. NCC is equivalent to SSD if we apply normalization to SSD. Specifically, to match images from two different modalities, the mutual information of images A and B is defined as:

$$I ( A , B ) = {\sum }_{a , b} p ( a , b ) log ( \frac{p ( a , b )}{p ( a ) p ( b )} ) . \qquad ( 1 )$$

The mutual information measures the distance between the joint distribution of the images intensity values p(a,b) and the joint distribution of the images intensity values when they are independent, p(a)p(b). It is a measure of dependence between two images. The assumption is that there is maximal dependence between the intensity values of the images when they are correctly aligned. Mis-registration will result in a decrease in the measure of mutual information. Therefore, larger mutual information implies more reliable registration.

The feature-based matching first determines a set of feature points in each image and then compares the corresponding feature descriptors. To match two image patches or features captured from two different viewing angles, a rigid model including scaling, rotation, etc. is estimated based on the correspondences. To match two images captured deforming objects, a non-rigid model including local deformation can be computed.

The number of feature points is usually much smaller than the number of pixels of a corresponding image. Therefore, the computational load for feature-based image matching is substantially less that for pixel-based image matching. However, it is still time consuming for pair-wise matching. Usually k-d tree, a well-known technique in this field, is utilized to speed up this procedure. Accordingly, feature-based image matching is widely used in the field. Nevertheless, the feature-based matching may not work well for images under some circumstances. In this case, the direct image matching can always be used as a fall back mode, or a combination of the above two approaches may be preferred.

Image matching techniques usually assume certain motion models. When the scenes captured by the camera consist of rigid objects, image matching based on either feature matching or pixel domain matching will work reasonably well. However, if the objects in the scene deform or lack of distinguishable features, it would make the image matching task very difficult. For capsule images captured during the course of travelling through the GI track, the situation is even more challenging. Not only the scenes corresponding to walls of the GI track deform while camera is moving and often are lack of distinguishable features, but also the scenes are captured with a close distance from the camera. Due to the close distance between objects and the camera, the often used linear camera model may fail to produce good match between different scenes. In addition, light reflection from near objects may cause over exposure for some parts of the object. Therefore, it is desirable to develop methods that can overcome the issues mentioned.

### SUMMARY OF INVENTION

A method of adaptively displaying images captured using a camera according to quality of image matching is disclosed. While image stitching provides an efficient viewing or examination of a large number of images, image stitching may cause noticeable artifacts particularly for images that do not fit camera models well. The present invention utilizes the quality of image matching to guide whether to stitch underlying images or not. Accordingly, an improved image reconstruction and a more visually pleasant viewing are achieved. In one embodiment, a plurality of images captured by the camera is received and the quality of image matching for each pair of images is determined. The quality of image matching is measured between two images to be matched. The image pair is declared as a high-confidence image pair or a low-confidence image pair according to the quality of image matching. The high-confidence image pairs are stitched into one or more larger composite pictures and displayed on a display device. On the other hand, the low-confidence image pairs are displayed as individual images without stitching.

The high-confidence matched images and the low-confidence unmatched images can be displayed on the display device in an interleaved manner, where the high-confidence matched images and the low-confidence unmatched images take turns to be displayed. The high-confidence matched images and the low-confidence unmatched images can also be displayed concurrently on the display device. In this case, the high-confidence matched images are displayed in one display area and the low-confidence unmatched images are displayed in another display area.

One aspect of the invention addresses various measurement of the quality of image matching. For example, the quality of image matching can be based on posterior probability corresponding to correct image matching given the features extracted and each matched feature pair is modeled as a binary random variable being an inlier or an outlier. In this case, the quality of image matching can be simply measured by counting the number of the matched feature pairs belonging to the inliers. The image pair is designated as a high-confidence matched image pair if the number of the features belonging to the inliers exceeds a threshold and, otherwise, the image pair is designated as a low-confidence unmatched image pair. The threshold is dependent on the probability of the feature being the inlier and the probability of the feature being the outlier.

The quality of image matching can also be based on pixel-domain measurement. For example, the quality of image matching can be based on the sum of squared distance (SSD) between two images to be matched. The image pair is designated as a high-confidence matched image pairs if the SSD is smaller than a threshold and, otherwise the image pair is designated as a low-confidence unmatched image pairs. Alternatively, the quality of image matching can be based on normalized cross correlation (NCC) or mutual information (MI) between two images to be matched. The image pair is designated as the high-confidence matched image pairs if the NCC or MI exceeds a threshold and, otherwise, the image pair is designated as the low-confidence unmatched image pairs.

For more complicated cases involving local deformation in addition to global transformation, the quality of image matching can be evaluated by combining image pyramids and free-form deformation such as B-spline deformation. Searching for good global matching can start from coarse level of the image pyramid by down-sampling the original image. In each pyramid level, global transformation can be estimated by averaging local shifting or doing exhaustive search. Such a global transformation will be refined in the next level of the image pyramid until the final level which is the original image. This procedure can also be iterated to compensate the outlier effects. After the global transformation is estimated, free-form deformation can be estimated in the overlap area by dividing the overlap area into a set of control points/grids. The output of the optimization function can be used as a confidence score to categorize image pairs into high-confidence matched image pairs or low-confidence unmatched image pairs.

### BRIEF DESCRIPTION OF THE DRAWINGS

FIG. 1 illustrates an exemplary high-confidence and low-confidence image pair's determination based on image matching, and displaying the high-confidence matched and unmatched low-confidence images in the same display area in an interleaved manner according to an embodiment of the present invention.

FIG. 2 illustrates an exemplary high-confidence and low-confidence image pair's determination based on image matching, and displaying the high-confidence matched and low-confidence unmatched images in respective display areas according to an embodiment of the present invention.

FIG. 3 illustrates an exemplary flowchart for a system for displaying images incorporating image stitching guided by quality of image matching according to an embodiment of the present invention.

### DETAILED DESCRIPTION OF THE INVENTION

It will be readily understood that the components of the present invention, as generally described and illustrated in the figures herein, may be arranged and designed in a wide variety of different configurations. Thus, the following more detailed description of the embodiments of the systems and methods of the present invention, as represented in the figures, is not intended to limit the scope of the invention, as claimed, but is merely a representative of selected embodiments of the invention. References throughout this specification to “one embodiment,” “an embodiment,” or similar language mean that a particular feature, structure, or characteristic described in connection with the embodiment may be included in at least one embodiment of the present invention. Thus, appearances of the phrases “in one embodiment” or “in an embodiment” in various places throughout this specification are not necessarily all referring to the same embodiment.

Furthermore, the described features, structures, or characteristics may be combined in any suitable manner in one or more embodiments. One skilled in the relevant art will recognize, however, that the invention can be practiced without one or more of the specific details, or with other methods, components, etc. In other instances, well-known structures, or operations are not shown or described in detail to avoid obscuring aspects of the invention. The illustrated embodiments of the invention will be best understood by reference to the drawings, wherein like parts are designated by like numerals throughout. The following description is intended only by way of example, and simply illustrates certain selected embodiments of apparatus and methods that are consistent with the invention as claimed herein.

As mentioned before, image matching may not work well for images under some circumstances, particularly for images captured using a capsule image through the human gastrointestinal (GI) track. Embodiments according to the present invention use a quality measure of image matching. According to the measured matching quality, a matching confidence level is determined. When the matching confidence level is good enough, the underlying images are stitched. Otherwise, the underlying images are not stitched. For example, if feature based image matching is used, image matching will be performed to match many correspondences. After matching, RANdom Sample Consensus (RANSAC) process will be used to select a set of inliers that are compatible with a transformation model between the images. RANSAC is a well-known technique in the field that is used to find a best transform among feature points between two images. In order to verify the match, a confidence score is calculated based on a probabilistic model.

For each pair of matching images, a subset of feature correspondences that are geometrically consistent (i.e., RANSAC inliers), and the remaining features are not consistent (i.e., RANSAC outliers). To verify the set of correspondences, the probabilities that the set of features is generated by correct image matching (i.e., inliers) or by false image matching (i.e., outliers) are evaluated. For a given image, the total number of features is denoted as n_{f} and the number of inliers is denoted as nᵢ, the event that image matching correctly/incorrectly is represented by a binary variable m ∈ {0,1}, where m=1 represents correct match and m=0 represents incorrect match. The event that the i^{th} feature correspondence is an inlier/outlier, represented by the binary variable f⁽ⁱ⁾ ∈ {0,1}, where f=1 represents inlier and f=0 represents outlier, is assumed to be an independent Bernoulli distribution. Accordingly, the probability of all features being inliers is Binomial distribution:

p(f^{(1:n}^{_{f}}⁾|m=1)=B(nᵢ; n_{f}, p₁), and (2)

p(f^{(1:n}^{_{f}}⁾|m=0)=B(nᵢ; n_{f}, p₀), (3)

where p₁ is the probability that a feature is an inlier given correct image matching, and p₀ is the probability that a feature is an inlier given false image matching. The total number of inliers, nᵢ is calculated according to nᵢ=Σᵢ₌₁ⁿ^{_{f}}f⁽ⁱ⁾. The posterior probability that image matching is correct can be evaluated using Bayes's Rule:

$$p ( m = 1 \vert {f}^{( 1 : {n}_{f} )} ) = p ( {f}^{( 1 : {n}_{f} )} \vert m = 1 ) p ( m = 1 ) \text{/} p ( {f}^{( 1 : {n}_{f} )} ) = 1 \text{/} ( 1 + p ( {f}^{( 1 : {n}_{f} )} \vert m = 0 ) p ( m = 0 ) \text{/} p ( {f}^{( 1 : {n}_{f} )} \vert m = 1 ) p ( m = 1 ) ) \qquad ( 4 )$$

Let the event of images matching correctly/incorrectly be a uniform prior (i.e., a prior probability distribution), p(m=0)=p(m=1). A criterion to accept image matching is based on whether p(m=1|f^{(1:n}^{_{f}}⁾)>pₘᵢₙ, where pₘᵢₙ is a minimum probability threshold used as a criterion to accept the image matching. Through a sequence of mathematically derivation, this condition is reduced to a likelihood ratio test:

$$\begin{matrix}B ( {n}_{i} ; {n}_{f} , {p}_{1} ) \text{/} B ( {n}_{i} ; {n}_{f} , {p}_{0} ) \begin{matrix}\begin{matrix}\begin{matrix}accept \\ >\end{matrix} \\ <\end{matrix} \\ reject\end{matrix} \frac{1}{1 \text{/} {p}_{min} - 1} , and & ( 5 ) \\ B ( {n}_{i} ; {n}_{f} , {p}_{1} ) = ( \begin{matrix}{n}_{f} \\ {n}_{i}\end{matrix} ) {{p}_{1}^{{n}_{i}} ( 1 - {p}_{1} )}^{{n}_{f} - {n}_{i}} & ( 6 )\end{matrix}$$

The values for pₘᵢₙ, p₁ and p₀ can be properly chosen according to image models or based on test data. The above decision process can be further simplified as the following testing:

nᵢ>g(n_{f}),

where g is a function of pₘᵢₙ, p₁ and p₀. In other words, after the values for pₘᵢₙ, p₁ and p₀ are determined, g can be determined. The decision process simply becomes counting the number of inlier, nᵢ. If the condition of eqn. (7) is satisfied, the image matching is verified and the registration is declared as confident registration. Otherwise the image matching is not verified and the registration has low confidence. In the above embodiment, the quality of image matching is measured in terms of the posterior probability that image matching is correct given the features extracted as shown in eqn. (4). If the quality of image matching is over a threshold (i.e., pₘᵢₙ), the image matching is verified. After further derivation, the decision process according to one embodiment of the present invention simply becomes counting the number of inlier, nᵢ, and comparing the result with a threshold. While the quality of image matching can be measured by counting the number of inlier, the quality of image matching can be measured by counting the number of outlier. In this case, if the number of outlier is less than a second threshold, the image matching is verified. Otherwise, image matching is not verified.

In another embodiment, the system uses non-feature based direct matching and calculates the sum of squared distance (SSD) as the measure of quality of image matching. The SSD between images A and B is defined as:

D_{SSD}(A,B|T)=Σ_{(x,y)}(A(x,y)−B(T(x,y)))², (7)

where (x,y) is the pixel in the overlap area, T is the transformation from image A to B. By carefully choosing a threshold Dmax, if Dssd(A,B|T)<Dmax, the image matching can be verified and the registration has high confidence. Otherwise the registration is not verified and the registration is not confident. In another embodiment normalized cross correlation (NCC) or mutual information (MI) can be used as a criterion to evaluate the quality of matching and compute the confidence score.

In another embodiment, in order to stitch two sequential images, each image of the pair will be down-sampled to create image pyramids first. From the coarse level, a global transformation will be estimated using exhaustive search within a pre-defined range. The resulting global transformation will be refined in the next level until the final level, which is the original image. After the global transformation is estimated, a free-form deformation transformation can be applied to the overlapped area to estimate the local deformation. The output of the optimization object function can be used as a criterion to evaluate the quality of matching and compute the confidence score.

In another embodiment, in order to stitch two sequential images, each image of the pair will be down-sampled to create image pyramids first. From the coarse level, a global transformation will be estimated by averaging the local transformation, which is computed by applying free-form deformation to the entire image. The resulting global transformation will be refined in the next level until the final level, which is the original image. Such a procedure will be iterated until the process converges to eliminate outlier effect. After the global transformation is estimated, a free-form deformation transformation can be applied to the overlapped area to estimate the local deformation. The output of the optimization object function can be used as a criterion to evaluate the quality of matching and compute the confidence score.

In another embodiment, more than two images can be stitched together with high confidence if and only if the following condition is true. Given the set of images i1,i2, . . . , iN, for each image ij (j=1,2, . . . N), we can find at least one image from the rest of images to match ij with high confidence. There might be multiple images matching ij with high confidence. Otherwise, it means ij cannot be stitched with the rest of images and will be removed from this image set. Above process can be repeated until no image will be removed from the image set. Then all the images in this set can be stitched together to form a large composite image. All the removed images will be displayed individually.

In one embodiment example, i1, i2, . . . iN are a sequence of images along time domain, where i1, i2, i3, i5, i6, i7, i8, i12 are found to find match with high confidence and are stitched together and displayed as composite image I1, while i4, i9, i10 and i11 could not and are displayed as single images. If i4 and i9 and i11 could find match with confidence while i10 could not, then i4, i9 and i11 are stitched together as a composite image I2 and displayed as such while i10 is displayed as single image in the video separately.

Sometimes the advantage of stitching too few images and displaying them in one composite image is outweighed by the disadvantage. For example the stitched images have arbitrary size while single image is fixed in dimensions and aspect ratio so looking at two stitched images in a composite image may not be as efficient in time compared with reading these two images in a video displayed at certain frame rate. A threshold T may be chosen to set the minimum number of images matched with high confidence before they are stitched and displayed as a composite image.

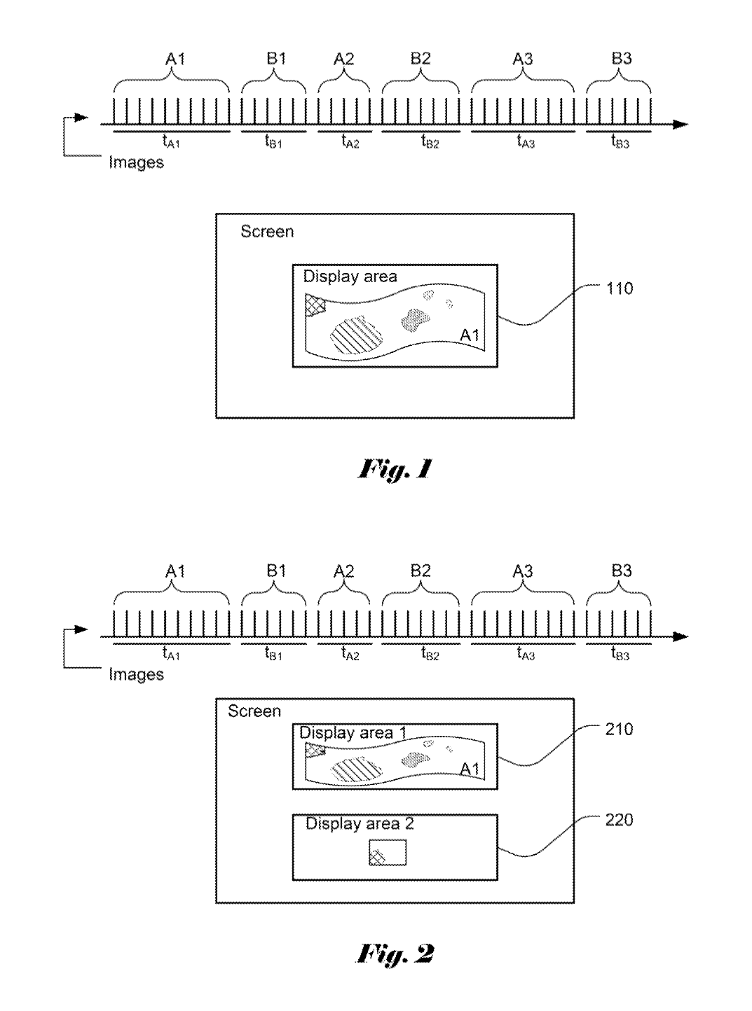

The quality of image matching disclosed above can be used to guide image stitching. When the quality of image matching is high, the registration can be declared to be confident. In one embodiment, the images are stitched to form a larger composite picture for images with high confidence even if there are discontinuities along transit time. For those images declared to be low confidence, the images are not stitched. The un-stitched images are treated as individual images or an image sequence and viewed as video. FIG. 1 illustrates one embodiment according to the present invention, where A1, A2 and A3 represent three groups of images with high confidence throughout the video. Each of A1, A2 and A3 corresponds to images in respective time periods t_{A1}, t_{A2} and t_{A3} having high confidence. The images within each group (i.e., A1, A2 or A3) are stitched into one or more larger composite pictures. B1, B2 and B3 correspond to images in respective time periods t_{B1}, t_{B2} and t_{B3} having low confidence. In one embodiment, images associated with A1, A2, A3 can be displayed in display area 110, and then then followed by images associated with B1, B2 and B3. FIG. 1 illustrates an instance that composite picture corresponding to group A1 is being displayed. The display order can be A1, A2 and A3 and then followed by B1, B2 and B3. The display may also follow the order of A1, B1, A2, B2, A3 and B3. When images associated with A1, A2, A3 are displayed, a stitched larger image or images can be used to allow a view to examine multiple images at the same time. When images associated with B1, B2 and B3 are displayed, the images will be treated as individual images and they can be displayed one by one manually or displayed as a video sequence at a desirable playback rate. The images are taken at uniform speed in FIG. 1. In another embodiment, images could be taken at non-uniform frame rate.

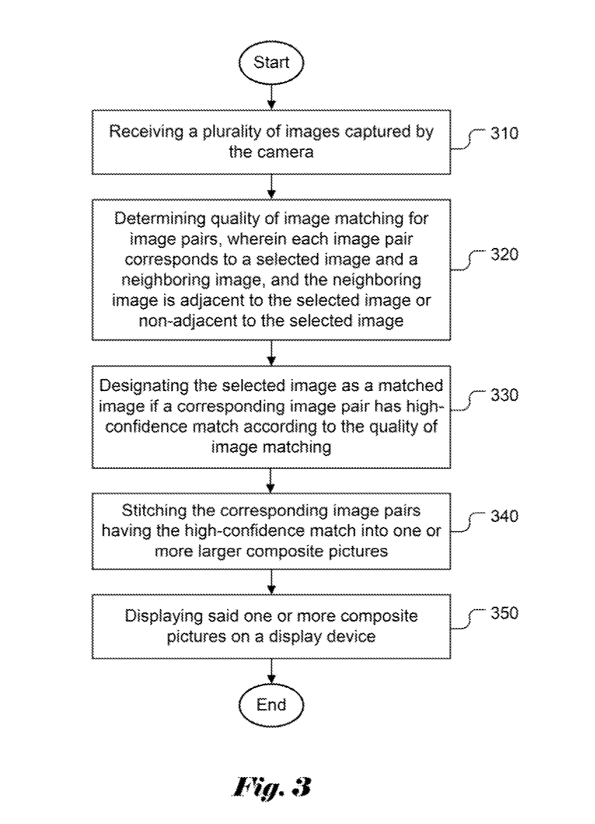

FIG. 2 illustrates another embodiment according to the present invention. Again, A1, A2 and A3 represent images with high confidence throughout the video. B1, B2 and B3 correspond to images having low confidence. There are two display areas, one is used to display A1, A2 and A3, the other one is used to display B1, B2 and B3. Two display areas (210 and 220) are used to display A1/A2/A3 and B1/B2/B3 separately. Images associated with A1, A2, A3 can be displayed as stitched larger composite in display area 210. Images associated with B1, B2 and B3 can be displayed as individual images. They can be displayed one by one manually or displayed in display area 220 as a video at a desirable play back rate.

FIG. 3 illustrates an exemplary flowchart of a system for displaying images incorporating image stitching guided by quality of image matching according to an embodiment of the present invention. A plurality of images captured by the camera is received as shown in step 310. The images may be retrieved from memory or received from a processor. The quality of image matching for each image is determined as shown in step 320. The quality of image matching is measured between two images to be matched. The image pair is then designated as a high-confidence matched image pair or a low-confidence unmatched image pair according to the quality of image matching as shown in step 330. The high-confidence images are stitched into one or more larger composite pictures as shown in step 340. The stitched larger composite pictures are displayed on a display device as shown in step 350.

While specific examples are directed to capsule images, the image stitching based on quality of image matching according to the present invention may also be applied to images of natural scenes captured at different viewing angles.

The invention may be embodied in other specific forms without departing from its spirit or essential characteristics. The described examples are to be considered in all respects only as illustrative and not restrictive. Therefore, the scope of the invention is indicated by the appended claims rather than by the foregoing description. All changes which come within the meaning and range of equivalency of the claims are to be embraced within their scope.

1. A method of displaying images of human gastrointestinal (GI) tract captured using a capsule camera travelling through the GI tract, the method comprising:
receiving a plurality of images captured by the capsule camera;
determining quality of image matching for image pairs, wherein each image pair corresponds to a selected image and a neighboring image, and the neighboring image is adjacent to the selected image or non-adjacent to the selected image;
designating the selected image as a matched image if a corresponding image pair has high-confidence match according to the quality of image matching;
designating the selected image as an unmatched image if all corresponding image pairs associated with the selected image have low-confidence match according to the quality of image matching, wherein the unmatched images are displayed on the display device as individual images or as a sequence without stitching;
stitching the corresponding image pairs having the high-confidence match into one or more larger composite pictures; and
displaying said one or more composite pictures on a display device.

2. (canceled)

3. (canceled)

4. The method of claim 1, wherein said one or more composite pictures and the unmatched images are displayed on the display device in an interleaved manner, wherein said one or more composite pictures are displayed during first periods and the unmatched images are displayed during second periods, and the first periods and the second periods are non-overlapping.

5. The method of claim 1, wherein said one or more composite pictures are displayed in a first display area on the display device and the unmatched images are displayed in a second display area on the display device.

6. The method of claim 1, wherein the quality of image matching is based on features extracted between each image pair.

7. The method of claim 6, wherein the quality of image matching is based on posterior probability corresponding to correct image matching under a condition of being provided the features extracted, wherein each feature is modeled as a binary random variable being an inlier or an outlier.

8. The method of claim 7, wherein the quality of image matching is measured by counting a number of the features belonging to the inliers, and the selected image is designated as the matched image if the number of the features belonging to the inliers exceeds a threshold and the selected image is designated as the unmatched image if the number of the features belonging to the inliers is below the threshold for all image pairs associated with the selected image.

9. The method of claim 8, wherein the threshold is dependent on a first probability corresponding to the feature being the inlier and a second probability corresponding to the feature being the outlier.

10. The method of claim 3, wherein the quality of image matching is based on a sum of squared distance (SSD), normalized cross correlation (NCC) or mutual information (MI) between each image pair.

11. The method of claim 10, wherein the selected image is designated as the matched image if the SSD is smaller than a threshold and the selected image is designated as the unmatched image if the SSD exceeds the threshold for all image pairs associated with the selected image.

12. The method of claim 10, wherein the selected image is designated as the matched image if the NCC or MI exceeds a threshold and the selected image is designated as the unmatched image if the NCC or MI is smaller than the threshold for all image pairs associated with the selected image.

13. The method of claim 1, wherein said determining the quality of image matching for the image pairs comprises:
generating image pyramids for each image of the corresponding image pairs;
estimating global transformation at a coarser level of the image pyramids by applying exhaustively search on coarser images of the image pyramids;
refining the global transformation at a finer level by using a global transformation result at the coarser level;
applying a local transformation based on free-form deformation to an overlapped area of the image pair to estimate local deformation, wherein parameters of the free form deformation are determined by optimizing an object function of the parameters; and
providing an output from the object function optimized as the quality of image matching for the image pairs.

14. The method of claim 1, wherein said determining the quality of image matching for the image pairs comprises:
generating image pyramids for each image of the corresponding image pairs;
applying local transformation computed by applying free-form deformation to each entire image pairs, wherein parameters of the free form deformation are determined by optimizing an object function of the parameters;
estimating global transformation by averaging the local transformation on coarser images of the image pyramids;
refining the global transformation at a finer level by using a global transformation result at a coarser level;
applying the free-form deformation to an overlapped area of the image pair to estimate local deformation after the global transformation is estimated at a final level of the image pyramids; and
providing an output from an object function optimized as the quality of image matching for the image pairs.

15. The method of claim 1, wherein said stitching the corresponding image pairs having the high-confidence match into one or more larger composite pictures is performed only if a number of corresponding image pair for one larger composite picture is greater than a threshold.

16. A system of displaying images of human gastrointestinal (GI) tract captured using a capsule camera travelling through the GI tract, the system comprising:
a display device; and
a processor coupled to the display device, wherein the processor is configured to:
receive a plurality of images captured by the capsule camera;
determine quality of image matching for image pairs, wherein each image pair corresponds to a selected image and a neighboring image, and the neighboring image is adjacent to the selected image or non-adjacent to the selected image;
designate the selected image as a matched image if a corresponding image pair has high-confidence match according to the quality of image matching; and
stitch the corresponding image pairs having the high-confidence match into one or more larger composite pictures; and
display said one or more composite pictures on the display device.

17. The system of claim 16, wherein the processor is configured further to designate the selected image as an unmatched image if all corresponding image pairs associated with the selected image have low-confidence match according to the quality of image matching.

18. The system of claim 17, wherein the unmatched images are displayed on the display device as individual images without stitching.

19. The system of claim 18, wherein said one or more composite pictures and the unmatched images are displayed on the display device in an interleaved manner, wherein said one or more composite pictures are displayed during first periods and the unmatched images are displayed during second periods, and the first periods and the second periods are non-overlapping.

20. The system of claim 18, wherein said one or more composite pictures are displayed in a first display area on the display device and the unmatched images are displayed in a second display area on the display device.

21. The system of claim 16, wherein the quality of image matching is based on features extracted between each image pair.

22. The system of claim 16, wherein the quality of image matching is based on a sum of squared distance (SSD), normalized cross correlation (NCC) or mutual information (MI) between each image pair.
